(19) United States
(12) Patent Application Publication
(10) Pub. No.: US 2015/0238169 A1
(43) Pub. Date: Aug. 27, 2015

(54) ULTRASONIC MEASUREMENT APPARATUS AND ULTRASONIC MEASUREMENT METHOD

(71) Applicant: SEIKO EPSON CORPORATION, Tokyo (JP)

(72) Inventor: Hiromitsu MIZUKAMI, Shiojiri-shi (JP)

(21) Appl. No.: 14/620,623

(22) Filed: Feb. 12, 2015

(30) Foreign Application Priority Data
Feb. 27, 2014 (JP) 2014-036408

(51) Int. Cl. A61B 8/08; A61B 5/08; A61B 8/02

(52) U.S. Cl. CPC A61B 8/5207; A61B 8/02; A61B 5/0816

(57) ABSTRACT

In an ultrasonic measurement apparatus, an ultrasonic sensor transmits an ultrasonic wave toward a blood vessel and receives a reflected wave. Then, in a processing unit of a main device, an ultrasonic measurement control section, a respiratory fluctuation component separation section, and a respiratory rate calculation section analyze the displacement of a vascular wall in a depth direction from the body surface using a received signal of the reflected wave, and detect the number of breaths per unit time using the analysis result. The unit time may be one minute or one second, for example.

### BACKGROUND

1. Technical Field

The present invention relates to an ultrasonic measurement apparatus that detects the number of breaths of a subject.

2. Related Art

As a method of detecting the number of breaths of a subject, for example, a technique of checking the respiratory status of the subject based on a pulse wave signal from the subject is known (refer to JP-A-2002-17696). In the technique disclosed in JP-A-2002-17696, the respiratory status of the subject is checked by emitting light to the subject and detecting a change in the amount of received light due to the flow of blood using an optical pulse wave sensor.

Incidentally, a bloodvessel repeats expansion/contraction because the organs or muscles around the blood vessel move due to breathing. Therefore, it is possible to calculate the number of breaths per unit time if the cycle of expansion/contraction of the blood vessel according to breathing is known.

### SUMMARY

An advantage of some aspects of the invention is to propose a new technique capable of correctly detecting the number of breaths of a subject.

A first aspect of the invention is directed to an ultrasonic measurement apparatus including: a transmission and reception unit that transmits an ultrasonic wave toward a blood vessel and receives a reflected wave; and a detection unit that analyzes displacement of a vascular wall in a depth direction from a body surface using a received signal of the reflected wave and detects the number of breaths per unit time using the analysis result.

As another aspect of the invention, the first aspect of the invention may be configured as an ultrasonic measurement method including: transmitting an ultrasonic wave toward a blood vessel and receiving a reflected wave; and analyzing displacement of a vascular wall in a depth direction from a body surface using a received signal of the reflected wave and detecting the number of breaths per unit time using the analysis result.

According to the first and the another aspects of the invention, it is possible to calculate the number of breaths per unit time by analyzing the displacement of the vascular wall in the depth direction from the body surface.

A second aspect of the invention is directed to the ultrasonic measurement apparatus according to the first aspect of the invention, wherein the detection unit detects the number of breaths by specifying a frequency of a respiratory fluctuation component by frequency analysis of the displacement of the vascular wall in the depth direction.

According to the second aspect of the invention, it is possible to calculate the number of breaths by specifying the frequency of the respiratory fluctuation component by frequency analysis of the displacement of the vascular wall in the depth direction.

A third aspect of the invention is directed to the ultrasonic measurement apparatus according to the second aspect of the invention, wherein the detection unit includes a heart rate calculation section that calculates a heart rate, and specifies a frequency of the respiratory fluctuation component by excluding a frequency corresponding to the heart rate from the frequency analysis result.

According to the third aspect of the invention, it is possible to specify the frequency of the respiratory fluctuation component after excluding the heart rate from the frequency analysis result.

A fourth aspect of the invention is directed to the ultrasonic measurement apparatus according to any one of the first to third aspects of the invention, wherein the detection unit detects the number of breaths based on displacement of one of a vascular front wall and a vascular rear wall in the depth direction.

According to the fourth aspect of the invention, it is possible to detect the number of breaths by analyzing the displacement of one of the vascular front wall and the vascular rear wall in the depth direction.

A fifth aspect of the invention is directed to the ultrasonic measurement apparatus according to the first aspect of the invention, wherein the detection unit detects the number of breaths based on a temporal change in a received signal strength in the vascular wall.

According to the fifth aspect of the invention, it is possible to detect the number of breaths from the temporal change in the received signal strength in the vascular wall.

A sixth aspect of the invention is directed to the ultrasonic measurement apparatus according to the first aspect of the invention, wherein the detection unit detects the number of breaths based on a temporal change in a vascular diameter that is determined by displacement of a vascular front wall in the depth direction and displacement of a vascular rear wall in the depth direction.

According to the sixth aspect of the invention, it is possible to detect the number of breaths from the temporal change in the vascular diameter.

A seventh aspect of the invention is directed to the ultrasonic measurement apparatus according to the sixth aspect of the invention, wherein the detection unit detects the number of breaths by performing frequency analysis of a vascular diameter variation from the temporal change in the vascular diameter, the vascular diameter variation indicating a temporal change in either a diastolic vascular diameter or a systolic vascular diameter.

According to the seventh aspect of the invention, it is possible to detect the number of breaths by extracting a vascular diameter variation indicating a temporal change in either the diastolic vascular diameter or the systolic vascular diameter and performing frequency analysis of the vascular diameter variation.

An eighth aspect of the invention is directed to the ultrasonic measurement apparatus according to any one of the first to seventh aspects of the invention, wherein the blood vessel is an artery.

According to the eighth aspect of the invention, it is possible to detect the number of breaths by analyzing the displacement of the blood vessel which is an artery in the depth direction.

### BRIEF DESCRIPTION OF THE DRAWINGS

The invention will be described with reference to the accompanying drawings, wherein like numbers reference like elements.

FIG. 1 is a diagram showing an example of the overall configuration of an ultrasonic measurement apparatus according to a first embodiment.

FIGS. 2A and 2B are diagrams for explaining the principle of detecting a vascular wall fluctuation.

FIG. 3 is a drawing showing an example of the vascular wall fluctuation waveform.

FIG. 4 is a diagram showing a result of FFT processing on the vascular wall fluctuation waveform.

FIG. 5 is a diagram showing a differential waveform of the vascular wall fluctuation waveform.

FIG. 6 is a block diagram showing an example of the functional configuration of the ultrasonic measurement apparatus according to the first embodiment.

FIG. 7 is a flowchart showing the procedure of the respiratory rate detection process in the first embodiment.

FIG. 8 is a diagram showing an example of the signal strength variation waveform.

FIG. 9 is a flowchart showing the procedure of the respiratory rate detection process in a modification example.

FIG. 10 is a diagram showing an example of the vascular diameter variation waveform.

FIG. 11 is a diagram showing a result of FFT processing on the diastolic vascular diameter variation waveform.

FIG. 12 is a block diagram showing an example of the functional configuration of an ultrasonic measurement apparatus according to a second embodiment.

FIG. 13 is a flowchart showing the procedure of the respiratory rate detection process in the second embodiment.

FIG. 14 is a diagram showing an example of the overall configuration of a blood vessel measurement apparatus in the modification example.

### DESCRIPTION OF EXEMPLARY EMBODIMENTS

Hereinafter, embodiments for implementing an ultrasonic measurement apparatus and an ultrasonic measurement method according to the invention will be described with reference to the accompanying diagrams. In addition, the invention is not limited by the embodiments described below, and applicable forms of the invention are not limited to the following embodiments. In the diagrams, the same components are denoted by the same reference numerals.

### First Embodiment

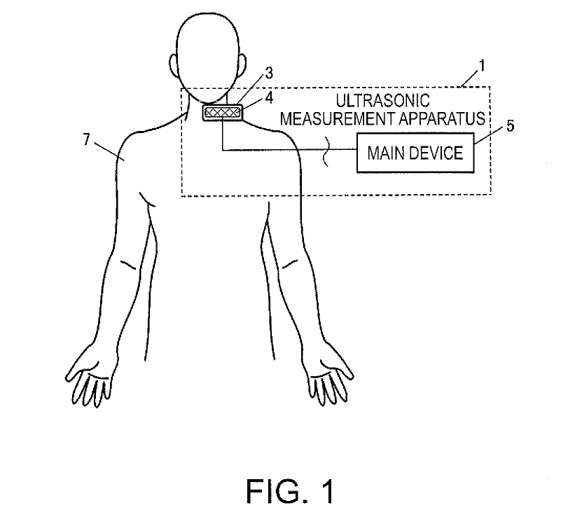

FIG. 1 is a diagram showing an example of the overall configuration of an ultrasonic measurement apparatus 1 according to a first embodiment. The ultrasonic measurement apparatus 1 according to the first embodiment detects the respiratory rate of the subject 7 based on a vascular wall fluctuation in the measurement target blood vessel (for example, carotid artery), and includes an ultrasonic probe 3 and a main device 5 as shown in FIG. 1. The ultrasonic probe 3 is for measuring the reflected wave of the ultrasonic wave, and includes an ultrasonic sensor 4 as a transmission and reception unit in which a plurality of ultrasonic transducers are arrayed in a two-dimensional manner, for example. The main device 5 acquires a vascular wall fluctuation by performing ultrasonic measurement using the ultrasonic probe 3, and calculates (estimates) the respiratory rate of the subject 7. Although the respiratory rate is described as the number of breaths per minute, the respiratory rate may be the number of breaths per unit time, and is not limited to the number of breaths per minute.

### Principle

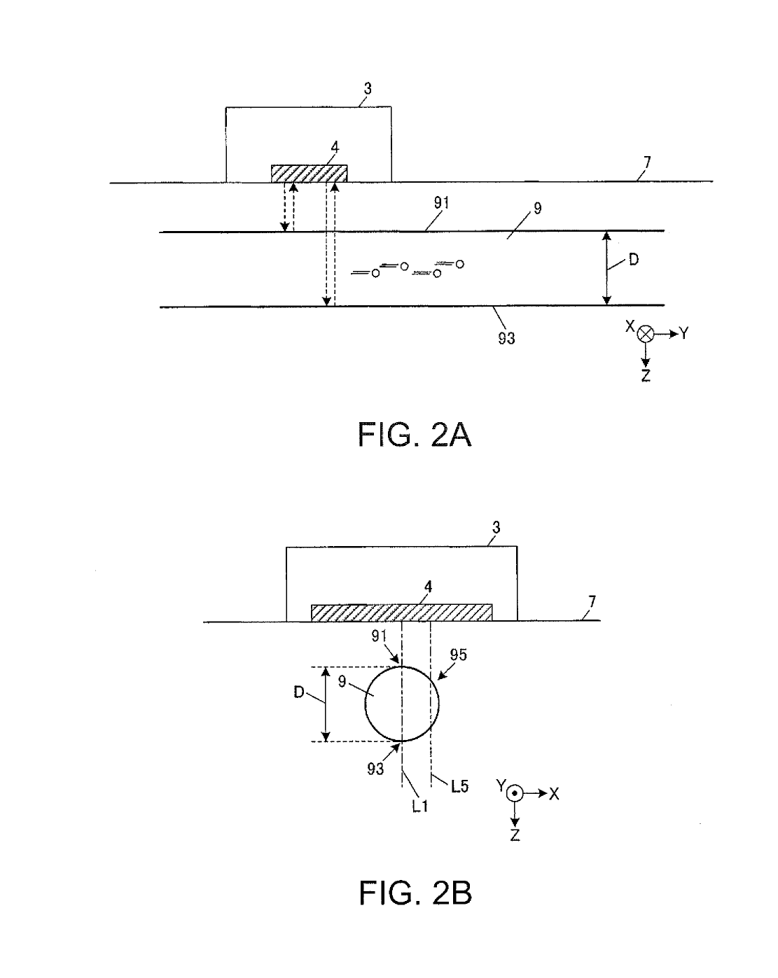

FIGS. 2A and 2B are diagrams for explaining the principle of acquiring a vascular wall fluctuation, where FIG. 2A schematically shows the cross section of the blood vessel 9 in a long-axis direction thereof (cross section of the blood vessel 9 in a traveling direction thereof) and FIG. 2B schematically shows the cross section of the blood vessel 9 in a short-axis direction thereof (surface of the blood vessel 9 perpendicular to the traveling direction). In FIGS. 2A and 2B, the long-axis direction of the blood vessel 9 is expressed as a Y direction, a depth direction from the body surface is expressed as a Z direction, and the short-axis direction of the blood vessel 9 perpendicular to the Y direction and the Z direction is expressed as an X direction.

In the ultrasonic measurement, the ultrasonic sensor 4 is positioned directly above the blood vessel 9 (for example, carotid artery) by placing the ultrasonic probe 3 on the body surface (here, the skin surface of the neck) of the subject 7. In addition, as indicated by the dashed arrow in FIG. 2A, the ultrasonic sensor 4 transmits a pulse signal or a burst signal of an ultrasonic wave having a frequency of several MHz to several tens of MHz toward the blood vessel 9 and receives a reflected wave from a vascular front wall 91 of the blood vessel 9 and a reflected wave from a vascular rear wall 93 of the blood vessel 9. On the other hand, the main device 5 generates reflected wave data relevant to the structure in the body of the subject 7 by performing amplification and signal processing on the received signal of the reflected wave that has been received by the ultrasonic sensor 4. This ultrasonic measurement is repeatedly performed at predetermined measurement periods (for example, at the frame rate of 300 frames per second to 500 frames per second).

Images of respective modes of a so-called A mode, B mode, M mode, and color Doppler mode are included in the reflected wave data. The A mode is a mode in which the amplitude (A-mode image) of the reflected wave is displayed on the assumption that the first axis indicates a distance from a predetermined body surface position in the depth direction (Z direction) and the second axis indicates a received signal strength of the reflected wave. The B mode is a mode in which a two-dimensional image (B-mode image) of the structure in the body visualized by converting the reflected wave amplitude (A-mode image), which is obtained while scanning the body surface position, into a brightness value is displayed.

The blood vessel 9 repeats approximately isotropic expansion and contraction according to the beating of the heart. Since the ultrasonic wave has a characteristic of being reflected greatly on the medium interface, a. reflected signal on the vascular wall appears strongly. However, the area of the surface perpendicular to the transmission direction of the ultrasonic wave can receive a stronger reflected wave. On the contrary, the area of the surface parallel to the transmission direction of the ultrasonic is more difficult to receive the reflected wave. For this reason, in the ultrasonic measurement, reflected waves from the vascular front wall 91 directly above the center of the blood vessel 9 and the vascular rear wall 93 directly below the center of the blood vessel 9 are detected strongly, but a reflected wave from the vascular transverse wall 95 is weak. Therefore, strong reflected waves relevant to the vascular front wall 91 and the vascular rear wall 93 appear in the reflected wave data.

Here, the main device 5 can perform so-called “tracking” that tracks a region of interest (tracking point) between different frames and calculates the displacement by setting a region of interest in the reflected wave data (for example, A-mode image) as a target.

In the first embodiment, the blood vessel 9 is detected from the B-mode image of the blood vessel short-axis cross section (XZ plane) using a method, such as pattern matching for detecting a circular shape that is a cross-sectional shape of the blood vessel, and the A-mode image corresponding to the reflected wave amplitude on the scanning line (line L1 shown by the one-dot chain line in FIG. 2B) passing near the center of the blood vessel 9 is selected as a. target. Then, a region of interest is set in the vascular front wall 91 in the selected A-mode image and tracking is performed to calculate the displacement in the depth direction from the body surface of the vascular front wall 91 due to beating or breathing, thereby acquiring a vascular wall fluctuation waveform. In addition, it is also possible to calculate a vascular diameter D for each frame by setting a region of interest not only in the vascular front wall 91 but also in the vascular rear wall 93 and performing tracking to calculate the displacement in the depth direction from the body surface of the vascular rear wall 93.

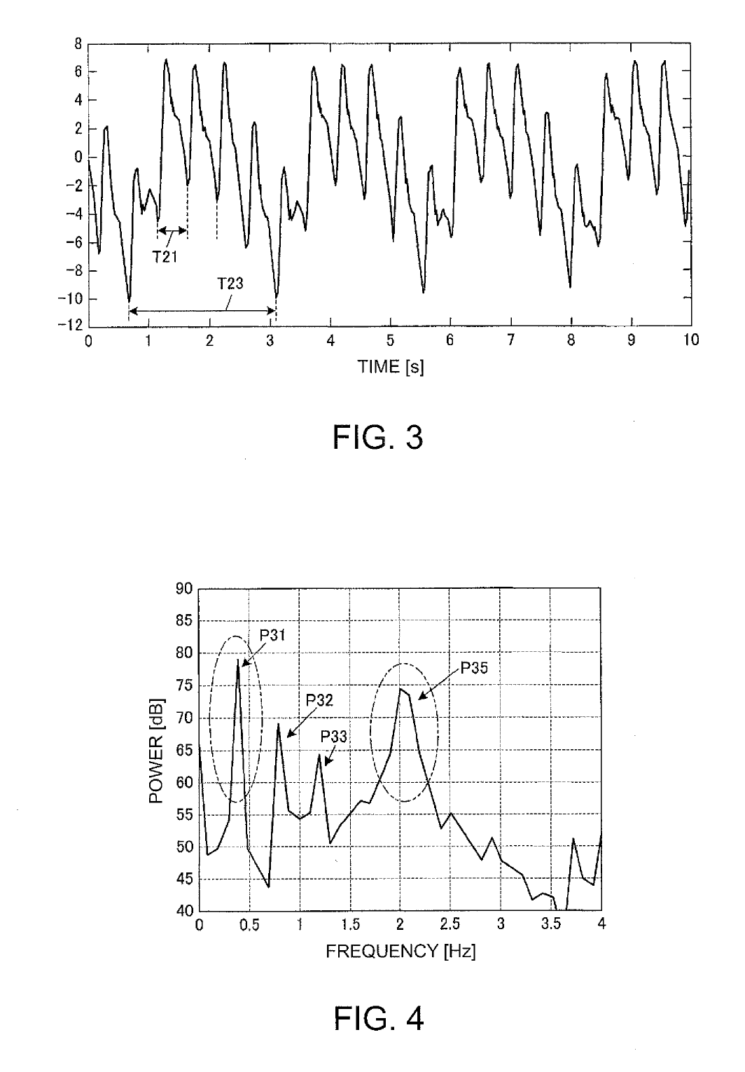

FIG. 3 is a diagram showing an example of the vascular wall fluctuation waveform. Not only does the blood vessel 9 repeat expansion/contraction according to the beating of the heart as described above, but also the blood vessel 9 expands/contracts because the organs or muscles around the blood vessel 9 move due to breathing. Generally, the heart beats multiple times while breathing once. Therefore, in the vascular wall fluctuation waveform, a fluctuation (beating fluctuation) due to beating appears with a short period T21, and a fluctuation (respiratory fluctuation) due to breathing appears with a period T23 longer than the period T21.

Therefore, a respiratory fluctuation component is separated out by performing frequency analysis by fast Fourier transform (FFT) processing on the vascular wall fluctuation waveform. FIG. 4 is a diagram showing a result of FFT processing performed on the vascular wall fluctuation waveform shown in FIG. 3. As a. result of the FFT processing, peaks of a plurality of frequency spectra are acquired. In FIG. 4, for example, a peak P31 surrounded by the one-dot chain line corresponds to a respiratory fluctuation component, and a peak P35 surrounded by the two-dot chain line corresponds to a beating fluctuation component.

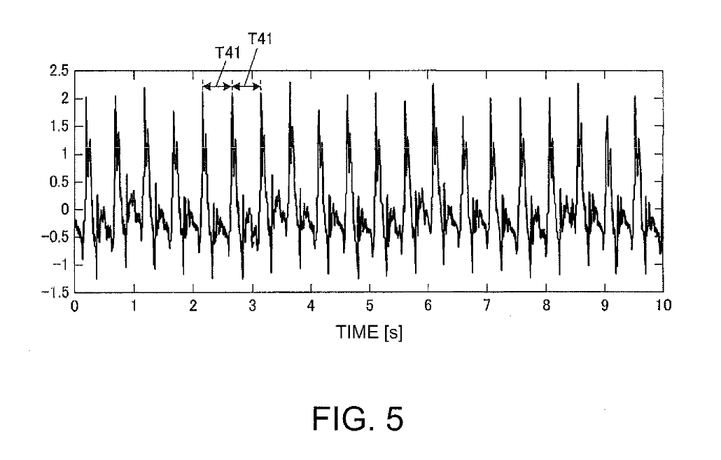

The peak P35 corresponding to the beating fluctuation component can be specified by calculating a beat frequency (heart rate) by differentiating the vascular wall fluctuation waveform. FIG. 5 is a diagram showing a differential waveform of the vascular wall fluctuation waveform shown in FIG. 3. A time T41 between the peaks of the differential waveform shown in FIG. 5 corresponds to the period (period T21 in FIG. 3) of beating fluctuation. Therefore, for example, by calculating the average value of the time T41 between the peaks as a period of beating fluctuation and calculating the frequency, it is possible to specify the peak (here, the peak P35) corresponding to the beating fluctuation component in the FFT processing result shown in FIG. 4.

If the peak P35 corresponding to the beating fluctuation component is specified as described above, the specified peak P35 is excluded, and then peaks having a frequency relationship of a fundamental wave and its integer harmonics (second harmonic, third harmonic, are selected from the remaining peaks. In FIG. 4, peaks P31, P32, and P33 are selected. Then, by specifying the frequency of the peak P31 of the fundamental wave as a frequency (breathing frequency) of the respiratory fluctuation component, it is possible to separate out the respiratory fluctuation component. Thereafter, a respiratory rate is calculated from the specified breathing frequency.

### Functional Configuration

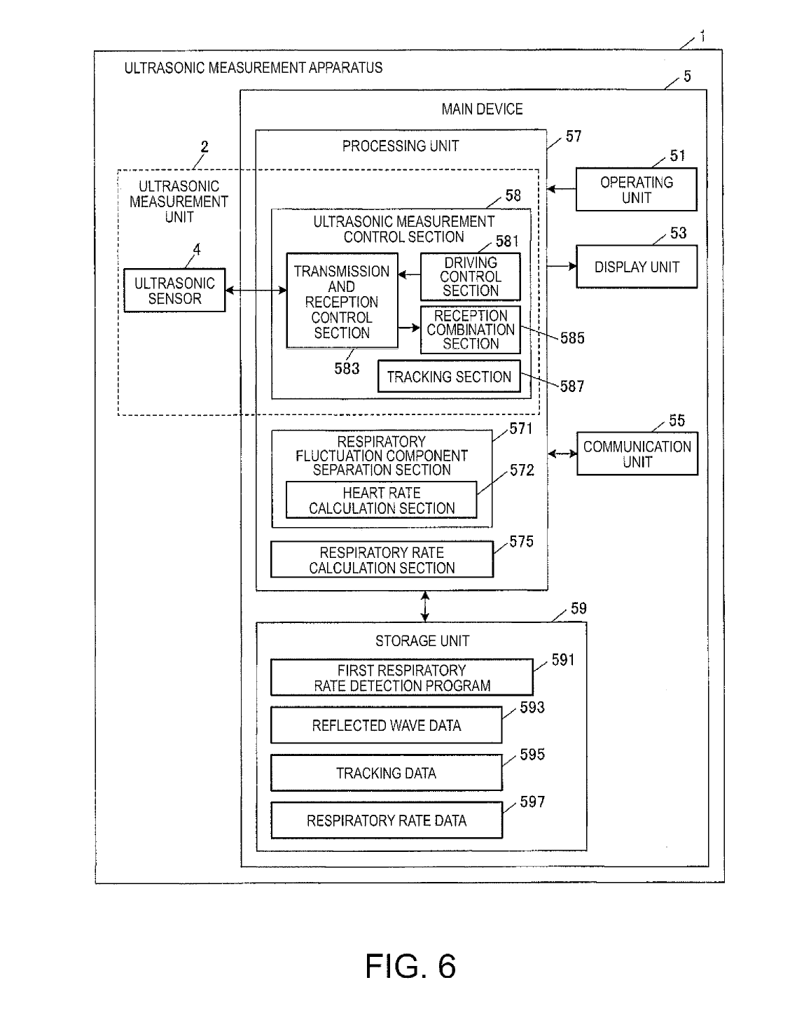

FIG. 6 is a block diagram showing an example of the main functional configuration of the ultrasonic measurement apparatus 1. As shown in FIG. 6, the main device 5 of the ultrasonic measurement apparatus 1 includes an operating unit 51, a display unit 53, a communication unit 55, a processing unit 57, and a storage unit 59. The main device 5 is connected to the ultrasonic sensor 4.

The operating unit 51 is realized by an input device, such as various switches including a button switch, a lever switch, and a dial switch, a touch panel, a track pad, or a mouse, and outputs an operation signal corresponding to an operation input to the processing unit 57.

The display unit 53 is realized by a display device, such as a liquid crystal display (LCD) or an electroluminescence display (EL display), and displays various screens based on a display signal input from the processing unit 57. The detected respiratory rate of the subject 7 and the like are displayed on the display unit 53. For example, according to the mode switching operation using the operating unit 51, a current respiratory rate display screen or a respiratory rate change display screen, which is a graph of the respiratory rate change based on the past logging data, is displayed.

The communication unit 55 is a communication device for transmitting and receiving data to and from the outside under the control of the processing unit 57. As a communication method of the communication unit 55, it is possible to apply various methods, such as a wired connection method using a cable based on a predetermined communication standard, a connection method using an intermediate device that also serves as a charger called a cradle, and a wireless connection method using wireless communication.

The processing unit 57 is an arithmetic device and a control device that performs overall control of the respective units of the ultrasonic measurement apparatus 1, and is realized by a microprocessor such as a central processing unit (CPU) or a graphics processing unit (GPU), an application specific integrated circuit (ASIC), or an integrated circuit (IC) memory. The processing unit 57 includes an ultrasonic measurement control section 58, a respiratory fluctuation component separation section 571, and a respiratory rate calculation section 575, and functions as a detection unit. In addition, each section of the processing unit 57 may be realized by hardware, such as an electronic circuit.

The ultrasonic measurement control section 58 forms an ultrasonic measurement unit 2 together with the ultrasonic sensor 4, and ultrasonic measurement is realized by the ultrasonic measurement unit 2. The ultrasonic measurement control section 58 can be realized by known techniques. For example, the ultrasonic measurement control section 58 includes a driving control section 581, a transmission and reception control section 583, a reception combination section 585, and a tracking section 587, and controls the transmission of an ultrasonic wave toward the blood vessel 9 and the reception of a reflected wave.

The driving control section 581 controls the transmission timing of the ultrasonic pulse from the ultrasonic sensor 4, and outputs a transmission control signal to the transmission and reception control section 583.

The transmission and reception control section 583 generates a pulse voltage according to the transmission control signal from the driving control section 581, and outputs the pulse voltage to the ultrasonic sensor 4. In this case, the output timing of the pulse voltage to each ultrasonic transducer is adjusted by performing transmission delay processing. In addition, the transmission and reception control section 583 performs amplification or filtering of the reflected wave signal input from the ultrasonic sensor 4, and outputs a processing result to the reception combination section 585.

The reception combination section 585 performs processing related to a so-called focus of a received signal by performing delay processing as necessary, thereby generating reflected wave data.

The tracking section 587 is a functional section that performs processing relevant to “tracking”, and tracks the position of a region of interest between frames of ultrasonic measurement based on the reflected wave data. For example, a process of detecting the position of the blood vessel 9 from the B-mode image, a process of setting a region of interest in the vascular front wall 91, a process of tracking the region of interest between different frames, and a process of calculating the displacement of the region of interest are performed. Thus, known functions, such as “phase difference tracking” or “echo tracking” are realized.

The respiratory fluctuation component separation section 571 specifies a breathing frequency from the vascular wall fluctuation waveform showing a temporal change in the region of interest (vascular front wall 91) tracked by the tracking section 587, and separates out a respiratory fluctuation component. The respiratory fluctuation component separation section 571 includes a heart rate calculation section 572 that calculates a beating frequency (heart rate) based on the vascular wall fluctuation waveform. In addition, the heart rate calculation section 572 may acquire the heart rate by receiving a signal indicating the beating of the subject 7 or a signal indicating the heart rate from the outside.

The respiratory rate calculation section 575 calculates the number of breaths of the subject 7 per unit time (for example, 1 minute) according to the breathing frequency as the respiratory rate. The unit time is not limited to 1 minute, and may be 1 second.

The storage unit 59 is realized by storage media, such as various integrated circuit (IC) memories including a read only memory (ROM), a flash ROM, and a random access memory (RAM), and a hard disk. In the storage unit 59, a program for operating the ultrasonic measurement apparatus 1 to realize various functions of the ultrasonic measurement apparatus 1, data used during the execution of the program, and the like are stored in advance or stored temporarily at each time of processing.

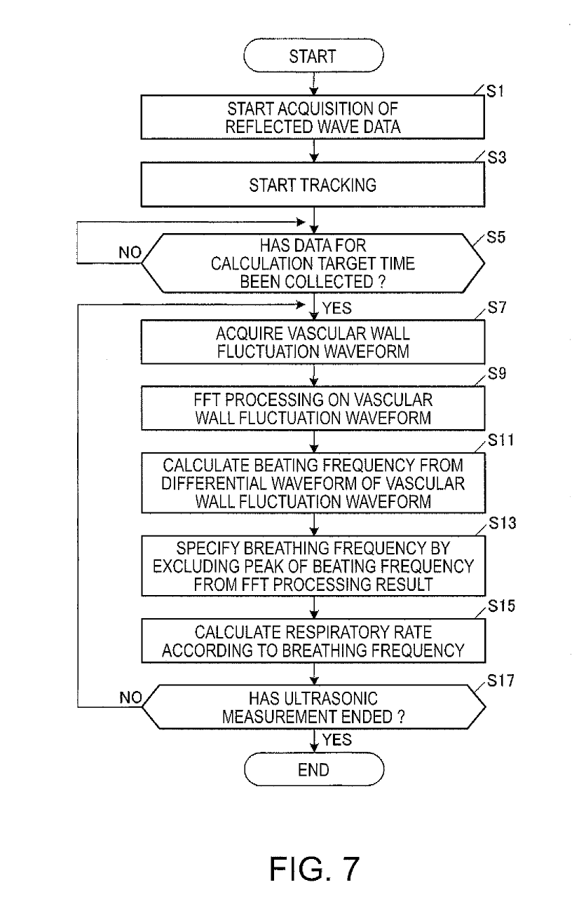

In addition, a first respiratory rate detection program 591 causing the processing unit 57 to function as the ultrasonic measurement control section 58, the respiratory fluctuation component separation section 571, and the respiratory rate calculation section 575 in order to perform the respiratory rate detection process (refer to FIG. 7), reflected wave data 593, tracking data 595, and respiratory rate data 597 are stored in the storage unit 59.

The reflected wave data 593 includes reflected wave data obtained by the ultrasonic measurement repeated for each frame. The reflected wave data 593 includes data of an A-mode image in each frame which is selected as a tracking target and in which a region of interest is set.

The tracking data 595 is the results data of tracking performed by the tracking section 587, and includes data of the displacement of the vascular front wall 91 in each frame that is selected as a region of interest and is tracked.

The respiratory rate data 597 includes a respiratory rate that is calculated every predetermined calculation period (for example, 10 seconds or 30 seconds) by the respiratory rate calculation section 575.

### Flow of the Process

FIG. 7 is a flowchart showing the procedure of the respiratory rate detection process. In addition, the process described herein can be realized when the processing unit 57 reads the first respiratory rate detection program 591 from the storage unit 59 and executes the first respiratory rate detection program 591. The respiratory rate detection process is started when the ultrasonic probe 3 is placed on the neck of the subject 7 and a predetermined measurement start operation is input.

In the respiratory rate detection process of the first embodiment, when the ultrasonic measurement control section 58 starts ultrasonic measurement first, the acquisition of reflected wave data is started by the reception combination section 585 (step S1), and tracking is started by the tracking section 587 (step S3). Then, only for the first time, a stand-by state occurs during the calculation target time to collect data for the calculation target time (step S5: No).

If the calculation target time has passed and the data for the calculation target time is collected (step S5: YES), the respiratory fluctuation component separation section 571 acquires the vascular wall fluctuation waveform by reading the latest tracking result for the calculation target time from the tracking data 595 (step S7), and performs FFT processing on the acquired vascular wall fluctuation waveform (step S9). Then, the heart rate calculation section 572 differentiates the vascular wall fluctuation waveform acquired in step S7, thereby calculating a beating frequency (heart rate) from the time between the peaks of the differential waveform (step S11).

Then, the respiratory fluctuation component separation section 571 specifies the frequency of the peak of the fundamental wave selected in the manner described above as a breathing frequency after excluding the peak of the beating frequency from the EFT processing result (step S13).

Then, the respiratory rate calculation section 575 calculates the number of breaths per minute according to the breathing frequency as a respiratory rate [number of times/minute] (step S15). The calculated respiratory rate is stored in the storage unit 59 as the respiratory rate data 597, and is displayed on the display unit 53 at an appropriate timing. Then, until the ultrasonic measurement is ended (step S17: No), the process returns to step S7 to repeat the processing described above.

As described above, according to the first embodiment, it is possible to acquire the vascular wall fluctuation waveform by setting a region of interest, for example, in the vascular front wall 91 and performing tracking to calculate the displacement in the depth direction from the body surface of the vascular front wall 91. Then, by performing the frequency analysis of the vascular wall fluctuation waveform and specifying a breathing frequency after excluding the peak of the beating frequency, it is possible to calculate the respiratory rate. Therefore, it is possible to realize a new technique capable of detecting the number of breaths of the subject 7 correctly.

In the first embodiment, the vascular wall fluctuation waveform is acquired by setting a region of interest in the vascular front wall 91 and performing tracking. In contrast, it is also possible to acquire the vascular wall fluctuation waveform by setting a region of interest in the vascular rear wall 93 and performing tracking.

Incidentally, the vascular wall is also displaced in a short-axis direction (X-direction) of the blood vessel since the blood vessel 9 expands/contracts according to beating or breathing. In addition, as described above with reference to FIGS. 2A and 2B, in the ultrasonic measurement, reflected waves from the vascular front wall 91 and the vascular rear wall 93 of the blood vessel 9 are detected strongly, but a reflected wave from the vascular transverse wall 95 is weak. Therefore, focusing on the reflected wave from the vascular transverse wall 95 on a specific scanning line (for example, a scanning line L5 in FIG. 2B), the received signal strength at the time of contraction of the blood vessel 9 is smaller than that at the time of expansion of the blood vessel 9. This is because the surface of the vascular transverse wall 95 at the time of contraction is closer to being parallel to the transmission direction of the ultrasonic wave than the surface of the vascular transverse wall 95 at the time of expansion is, and accordingly, the reflected wave from the vascular transverse wall 95 is weak. Therefore, the respiratory rate of the subject 7 may be detected based on a temporal change in the received signal strength of the reflected wave from the vascular transverse wall 95.

In this modification example, an A-mode image of the scanning line passing through the vascular transverse wall 95 is selected as a target. Then, a region of interest is set in the vascular transverse wall 95 of the selected A-mode image and tracking is performed, and a temporal change in the received signal strength in the region of interest in each frame is acquired as a signal strength variation waveform.

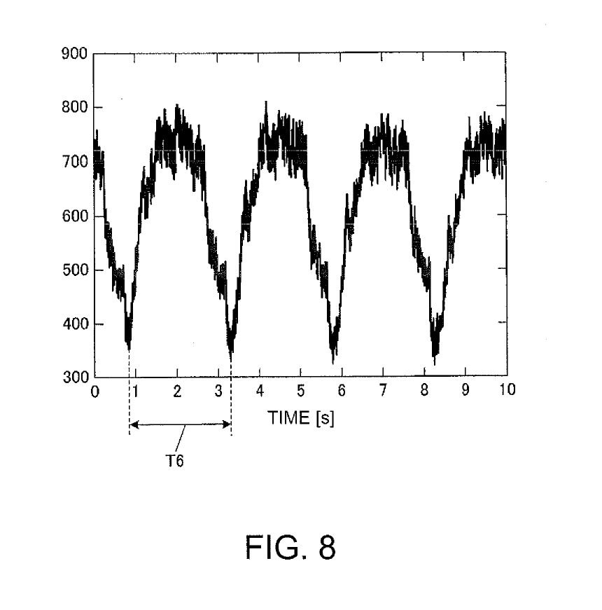

FIG. 8 is a diagram showing an example of a signal strength variation waveform. The signal strength variation waveform repeats fine beating fluctuations, thereby drawing a period of breathing as a whole. Accordingly, by calculating the average value of time T6 between the minimum values of each period as a period of breathing, it is possible to calculate the respiratory rate. In addition, the average value of the time between the maximum values of each period may be calculated as a period of breathing.

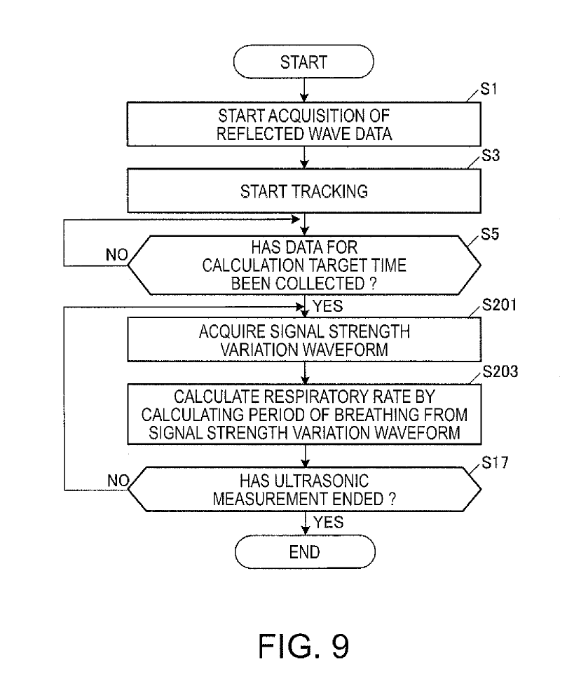

FIG. 9 is a flowchart showing the procedure of the respiratory rate detection process in this modification example. In addition, the same processes as in the first embodiment are denoted by the same reference numerals.

In the respiratory rate detection process of this modification example, data of the calculation target time is collected for the first time (step S5: YES), and then a signal strength variation waveform is acquired using the latest tracking result for the calculation target time that is read from the tracking data 595 (step S201). For example, a received signal strength in a region of interest of the A-mode image set as a tracking target according to the tracking result for the calculation target time is read from the reflected wave data 593. Then, the average value of the read received signal strengths in the region of interest is calculated for each frame, and a. temporal change in the calculated average value is acquired as a signal strength variation waveform.

After the signal strength variation waveform is acquired, a respiratory rate is calculated by calculating the period of breathing from the time between the minimum values of each period that the signal strength variation waveform draws (step S203). Then, the process proceeds to step S17.

According to this modification example, it is possible to set a region of interest, for example, in the vascular transverse wall 95 and perform tracking to acquire, as a signal strength variation waveform, a temporal change in the received signal strength of the reflected wave from the vascular transverse wall 95 in each frame according to displacement in the depth direction from the body surface of the vascular transverse wall 95. Then, by calculating the period of breathing from the signal strength variation waveform, it is possible to calculate a respiratory rate. Therefore, it is possible to realize anew technique capable of detecting the number of breaths of the subject 7 correctly.

### Second Embodiment

In a second embodiment, the respiratory rate of the subject 7 is detected based on a temporal change in the vascular diameter due to beating or breathing. In addition, the same portions as in the first embodiment are denoted by the same reference numerals.

### Principle

First, a region of interest is set in both the vascular front wall 91 and the vascular rear wall 93, and tracking is performed. Then, the vascular diameter D is calculated for each frame in the manner described above with reference to FIGS. 2A and 2B, and a vascular diameter variation waveform showing a temporal change in the vascular diameter D is acquired.

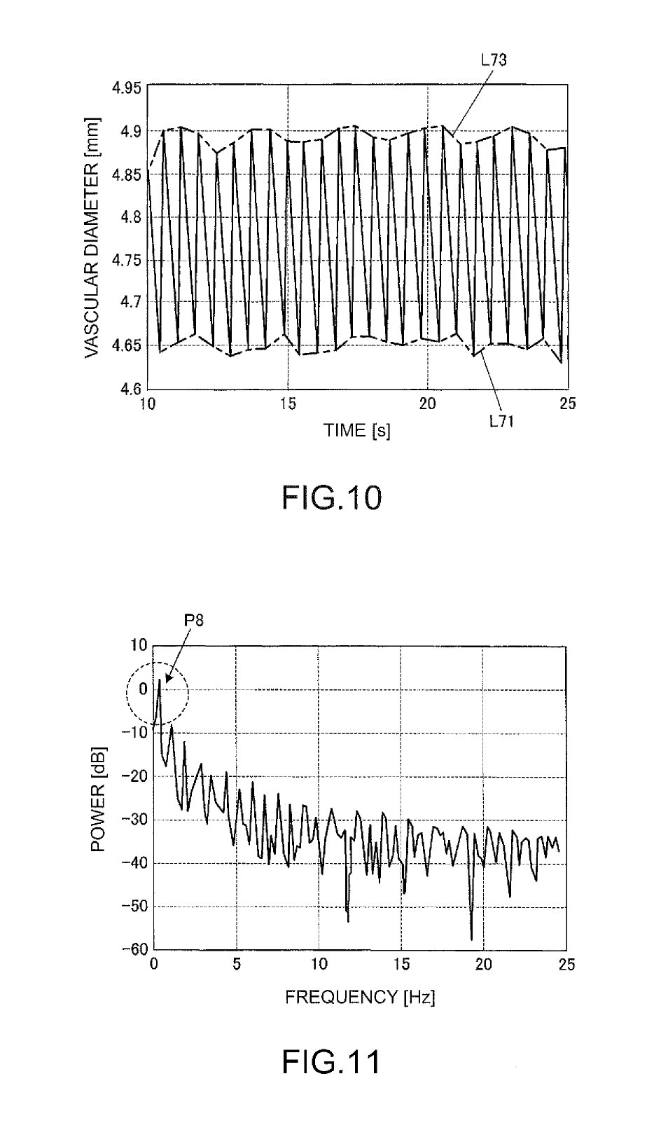

FIG. 10 is a diagram showing an example of the vascular diameter variation waveform. As shown in FIG. 10, the vascular diameter varies greatly due to expansion and contraction of the heart that are repeated every beat (every cardiac beat), and the vascular diameter is large in systole and small in diastole. Therefore, if only the vascular diameter in one of the systole and the diastole is extracted, it is possible to separate out a respiratory fluctuation component by removing a. beating fluctuation component from the vascular diameter variation waveform.

For example, a. diastolic vascular diameter variation waveform L71 showing a temporal change in the diastolic vascular diameter, which is shown by the one-dot chain line in FIG. 10, is generated by extracting (sampling) only the vascular diameter in the diastole. In this case, since the sampling times of the sampled diastolic vascular diameters are not necessarily equally spaced, it is preferable to appropriately perform a resampling process for equally spaced data. In addition, it is also possible to generate the diastolic vascular diameter variation waveform L71 by applying a technique relevant to envelope detection. In addition, as shown by the two-dot chain line in FIG. 10, a systolic vascular diameter variation waveform L73 showing a temporal change in the systolic vascular diameter may be generated by extracting only the vascular diameter in the systole, and a respiratory fluctuation component can be separated by performing subsequent processing in the same manner.

After the diastolic vascular diameter variation waveform is generated, a respiratory fluctuation component is separated out by performing frequency analysis by FFT processing on the generated diastolic vascular diameter variation waveform. FIG. 11 is a diagram showing the FFT processing result of the diastolic vascular diameter variation waveform L71 shown by the one-dot chain line in FIG. 10. From the FFT processing result, by specifying the frequency of a peak P8 of the highest spectrum surrounded by the dashed line in FIG. 11 as a breathing frequency, it is possible to separate out the respiratory fluctuation component. Thereafter, a respiratory rate [number of times/minute] is calculated from the specified breathing frequency. In the example shown in the diagrams, the frequency of the peak P8 is 0.39 [Hz]. Accordingly, the respiratory rate can be calculated as 0.39×60=18 [number of times/minute].

### Functional Configuration

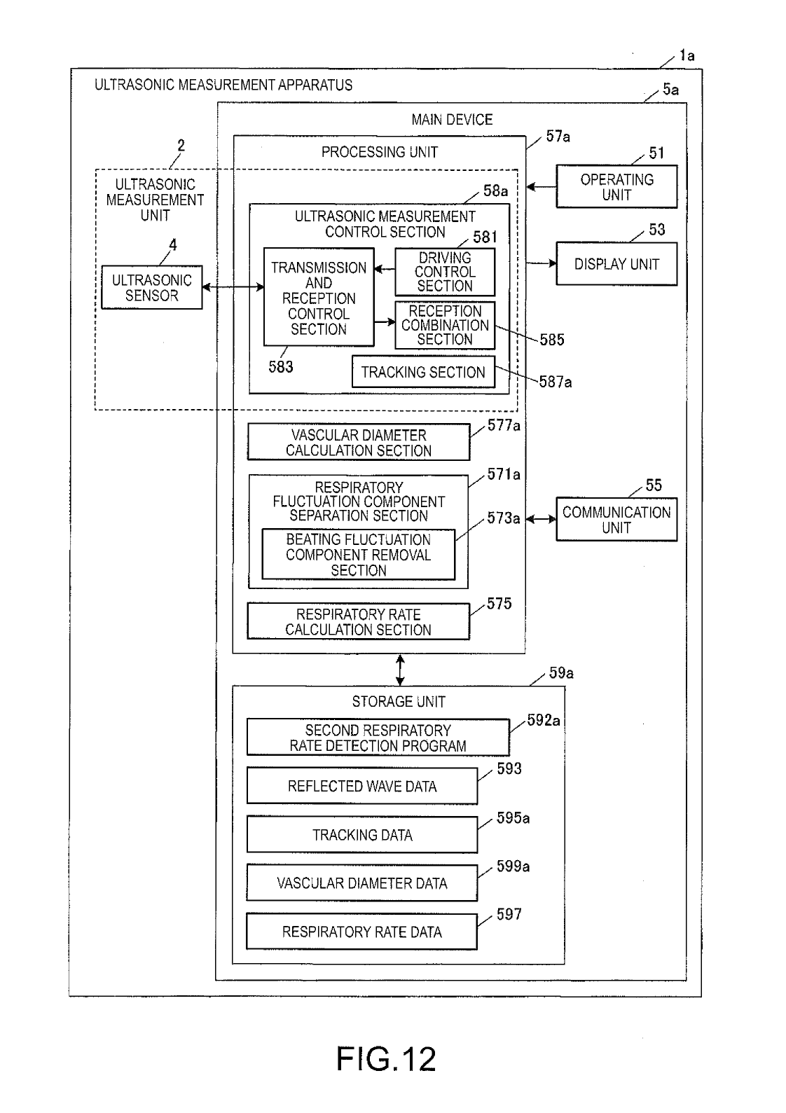

FIG. 12 is a block diagram showing an example of the main functional configuration of an ultrasonic measurement apparatus 1a according to the second embodiment. As shown in FIG. 12, a main device 5a of the ultrasonic measurement apparatus 1a includes an operating unit 51, a display unit 53, a communication unit 55, a processing unit 57a, and a storage unit 59a. The main device 5a is connected to the ultrasonic sensor 4, thereby forming the ultrasonic measurement apparatus 1a.

In the second embodiment, the processing unit 57a includes an ultrasonic measurement control section 58a, a vascular diameter calculation section 577a, a respiratory fluctuation component separation section 571a, and a respiratory rate calculation section 575.

In the ultrasonic measurement control section 58a, a tracking section 587a sets a region of interest in the vascular front wall 91 and the vascular rear wall 93 of the target A-mode image and calculates a displacement for each region of interest by tracking each region of interest between different frames.

The vascular diameter calculation section 577a calculates a vascular diameter for each frame from the displacement of the vascular front wall 91 and the displacement of the vascular rear wall 93 obtained by tracking the region of interest with the tracking section 587a.

The respiratory fluctuation component separation section 571a generates a vascular diameter variation waveform showing a temporal change in the vascular diameter calculated for each frame by the vascular diameter calculation section 577a, and separates out a respiratory fluctuation component by specifying a breathing frequency from the vascular diameter variation waveform. The respiratory fluctuation component separation section 571a includes a beating fluctuation component removal section 573a that removes a beating fluctuation component by generating a diastolic vascular diameter variation waveform from a vascular diameter variation waveform.

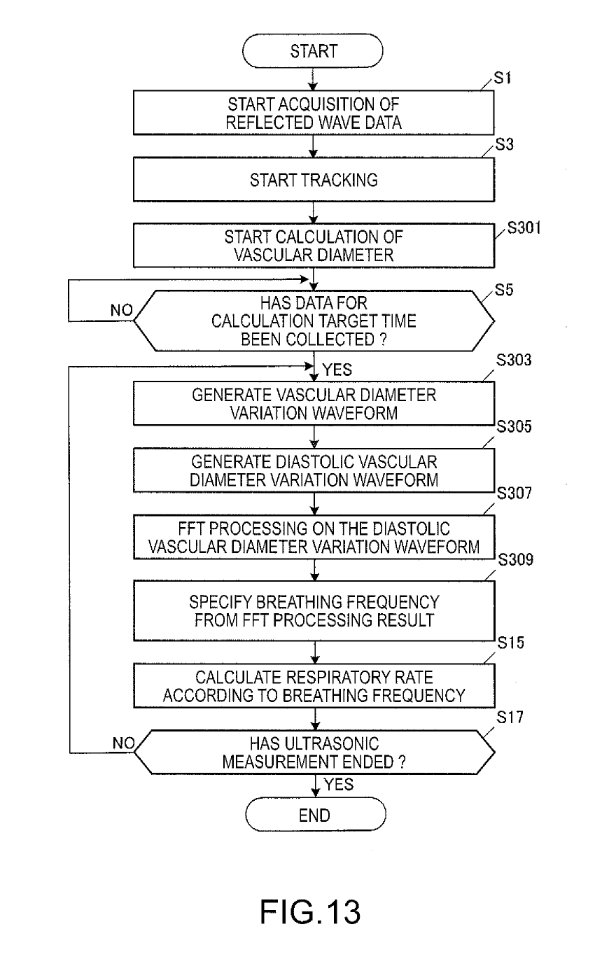

In addition, a second respiratory rate detection program 592a causing the processing unit 57a to function as the ultrasonic measurement control section 58a, the vascular diameter calculation section 577a, the respiratory fluctuation component separation section 571a, and the respiratory rate calculation section 575 in order to perform the respiratory rate detection process (refer to FIG. 13), reflected wave data 593, tracking data 595a, vascular diameter data 599a, and respiratory rate data 597 are stored in the storage unit 59a.

The tracking data 595a includes the displacement of the vascular front wall 91 and the displacement of the vascular rear wall 93 in each frame that are selected as regions of interest and are tracked. The vascular diameter data 599a includes a vascular diameter calculated for each frame by the vascular diameter calculation section 577a.

### Flow of the Process

FIG. 13 is a flowchart showing the procedure of the respiratory rate detection process. In addition, the process described herein can be realized when the processing unit 57a reads the second respiratory rate detection program 592a from the storage unit 59a and executes the second respiratory rate detection program 592a.

In the respiratory rate detection process of the second embodiment, the acquisition of reflected wave data is started in step S1, tracking is started in step S3, and then the vascular diameter calculation of the vascular diameter calculation section 577a is started (step S301). Then, only for the first time, a stand-by state occurs during the calculation target time to collect data for the calculation target time (step S5: No).

If the calculation target time has passed and the data for the calculation target time is collected (step S5: YES), the respiratory fluctuation component separation section 571a generates a vascular diameter variation waveform by reading the latest vascular diameter for the calculation target time from the vascular diameter data 599a (step S303).

Then, the beating fluctuation component removal section 573a removes a beating fluctuation component by generating a diastolic vascular diameter variation waveform by sampling only the vascular diameter in the diastole from the vascular diameter variation waveform generated in step S303 (step S305).

Then, the respiratory fluctuation component separation section 571a performs FFT processing on the diastolic vascular diameter variation waveform (step S307), and specifies the frequency of the peak of the highest spectrum from the FFT processing result as a breathing frequency (step S309). Then, the process proceeds to step S15.

As described above, according to the second embodiment, by setting a region of interest in both of the vascular front wall 91 and the vascular rear wall 93 and performing tracking, it is possible to acquire the vascular diameter variation waveform showing a temporal change in the vascular diameter determined by the vascular front wall 91 and the vascular rear wall 93. Then, by extracting only the diastolic vascular diameter from the vascular diameter variation waveform, it is possible to generate a diastolic vascular diameter variation waveform from which a beating fluctuation component has been removed. By specifying a breathing frequency from the diastolic vascular diameter variation waveform, it is possible to calculate the respiratory rate. Therefore, it is possible to correctly detect the number of breaths of the subject 7.

In addition, although the carotid artery has been exemplified as a. measurement target blood vessel in each of the embodiments described above, other types of blood vessels may also be used as measurement target blood vessels. However, it is preferable to use an artery having a larger fluctuation due to beating or breathing than a vein.

In addition, the ultrasonic measurement apparatus described in each of the above embodiments may be made to have a function of measuring blood pressure in a non-pressure method using an ultrasonic wave, so that the respiratory rate is detected simultaneously with the measurement of blood pressure. It is known that breathing affects a blood pressure variation. On the other hand, the vascular diameter and blood pressure can be associated with each other by certain nonlinear correlation characteristics.

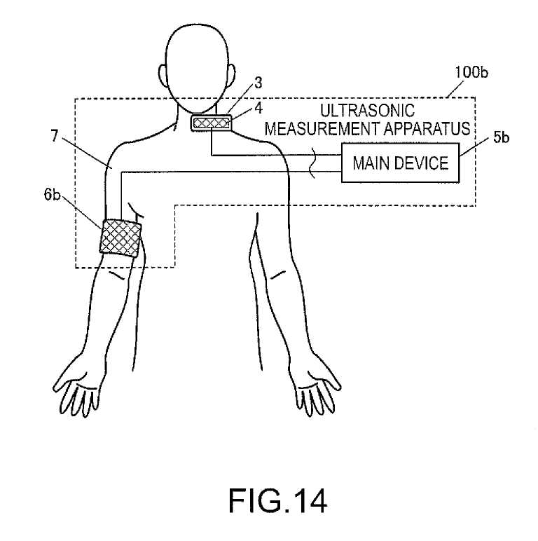

FIG. 14 is a diagram showing an example of the overall configuration of an ultrasonic measurement apparatus 100b in a modification example. The ultrasonic measurement apparatus 100b in this modification is formed integrally with a pressure sphygmomanometer, and includes an ultrasonic probe 3, a cuff 6b, and a main device 5b as shown in FIG. 14.

The main device 5b has a configuration necessary for calculating (estimating) blood pressure based on the vascular diameter of the measurement target blood vessel (for example, carotid canal) in addition to the configuration of the main device described in each of the embodiments described above.

Here, the correlation characteristics between the vascular diameter and blood pressure described above can be expressed by the correlation equation shown in the following Expression (1) from the pressure applied to the blood vessel and the vascular diameter at the time of each blood pressure. In the following Expression (1), “Ps” is systolic blood pressure (highest blood pressure), and “Pd” is a diastolic blood pressure (lowest blood pressure). “Ds” is a systolic vascular diameter that is a vascular diameter at the time of systolic blood pressure, and “Dd” is a diastolic vascular diameter that is a vascular diameter at the time of diastolic blood pressure. In addition, “β” is a vascular elasticity index value called a stiffness parameter.

P=Pd·exp[β(D/Dd−1)] (1)

Here, β=ln(Ps/Pd)/(Ds/Dd−1) (2)

When calculating the blood pressure from the vascular diameter using the correlation equation of the above Expression (1), it is necessary to measure blood pressure for calibration separately from the vascular diameter. The cuff 6b is a pressure cuff for blood pressure measurement at the time of calibration, and the ultrasonic measurement apparatus 100b performs pressurizing blood pressure measurement using the cuff 6b at the time of calibration. In FIG. 14, a cuff that is wrapped around the upper arm of the subject 7 to measure the blood pressure of the brachial artery is shown. The cuff 6b is removed from the subject 7 after calibrating the ultrasonic measurement apparatus 100b. Thereafter, the ultrasonic probe 3 is used alone, and the blood pressure of the subject 7 is measured in a non-pressure method.

The entire disclosure of Japanese Patent Application No. 2014-036408, filed on Feb. 27, 2014 is expressly incorporated by reference herein.

What is claimed is:

1. An ultrasonic measurement apparatus, comprising:
a transmission and reception unit that transmits an ultrasonic wave toward a blood vessel and receives a reflected wave; and
a detection unit that analyzes displacement of the blood vessel using a received signal of the reflected wave and detects the number of breaths per unit time using the analysis result.

2. The ultrasonic measurement apparatus according to claim 1,
wherein the detection unit detects the number of breaths by specifying a frequency of a respiratory fluctuation component by frequency analysis of the displacement of the blood vessel.

3. The ultrasonic measurement apparatus according to claim 2,
wherein the detection unit includes a heart rate calculation section that calculates a heart rate, and specifies a frequency of the respiratory fluctuation component by excluding a frequency corresponding to the heart rate from the frequency analysis result.

4. The ultrasonic measurement apparatus according to claim 1,
wherein the detection unit detects the number of breaths based on displacement of one of a vascular front wall and a vascular rear wall.

5. The ultrasonic measurement apparatus according to claim 1,
wherein the detection unit detects the number of breaths based on a temporal change in a received signal strength in the vascular wall.

6. The ultrasonic measurement apparatus according to claim 1,
wherein the detection unit detects the number of breaths based on a temporal change in a vascular diameter that is determined by displacement of a vascular front wall and displacement of a vascular rear wall.

7. The ultrasonic measurement apparatus according to claim 6,
wherein the detection unit detects the number of breaths by performing frequency analysis of a vascular diameter variation from the temporal change in the vascular diameter, the vascular diameter variation indicating a temporal change in either a diastolic vascular diameter or a systolic vascular diameter.

8. The ultrasonic measurement apparatus according to claim 1,
wherein the blood vessel is an artery.

9. An ultrasonic measurement method, comprising:
transmitting an ultrasonic wave toward a blood vessel and receiving a reflected wave; and
analyzing displacement of the blood vessel using a received signal of the reflected wave and detecting the number of breaths per unit time using the analysis result.
